(19) United States
(12) Patent Application Publication
(10) Pub. No.: US 2024/0074371 A1
(43) Pub. Date: Mar. 7, 2024

(54) An embryo rescue and in vitro herbicidal selection method for sunflower

(71) Applicant: Syngenta Crop Protection AG, Basel (CH)

(72) Inventors: Reijie CUI, Beijing (CN); Yinghui DAN, Raleigh, NC (US); Manuel Benito SAINZ, Fuquay Varina (BR); Huaibing JIN, Beijing (CN)

(73) Assignee: Syngenta Crop Protection AG, Basel (CH)

(21) Appl. No.: 18/271,994

(22) PCT Filed: Dec. 16, 2021
(86) PCT No.: PCT/CN2021/138762

§ 371 Date: Jul. 12, 2023

(30) Foreign Application Priority Data
Jan. 15, 2021 (CN) 202110052428.0

(51) Int. Cl. A01H 1/00; A01H 4/00; A01H 6/14

(52) U.S. Cl. CPC A01H 1/123; A01H 4/005; A01H 6/1464

(57) ABSTRACT

Sunflower is an important oilseed crop. Typically, new cultivars with improved agronomic traits are developed through conventional breeding program. Here, we describe a method of sunflower embryo rescue to accelerate trait introduction into plants. Further, weeds are a significant issue for the sunflower crop. However, there are not many selective herbicides for sunflower. Here, using the embryo rescue methodology, we further describe in vitro herbicidal section methodologies.

### FIELD OF THE INVENTION

This invention relates to the fields of embryo rescue and in vitro herbicidal selection methodologies for sunflower.

### BACKGROUND

Sunflower (Helianthus annuus) is one of the most important oilseed crops in the world. Conventional breeding has resulted in cultivars with improved agronomic traits. Modern commercial cultivars with valuable agronomic performance are typically hybrids of male and female inbred lines.

A conventional sunflower breeding program involves several breeding cycles. Embryo rescue (ER) technology can be used to accelerate the introduction of traits into commercial inbred lines through life cycle shortening (LCS). Previous published reports have addressed ER technology primarily in the context of wide crosses with exotic Helianthus species that are the source of many agronomically useful sunflower traits. Media components, including basal media, sucrose concentrations, vitamins and plant growth regulators (hormones), are major factors in the development of ER protocols in different species (Lulsdorf et al. 2013).

One issue for sunflower are weeds and the significant losses in yield that competition from weeds can cause. Competition from the weeds can reduce the amount of moisture, nutrients, light, and/or space that the sunflower receives. Sunflower yield losses due to weeds has been reported as high as 70%. For example, broomrape (Orobanche) is an obligate parasite that parasitizes sunflower. Broomrape feeds on the roots of the plant and sprouts, producing a large quantity of seeds. Herbicide are an important control component of broomrape considering genetic resistance to the parasite is incomplete. The combination of herbicides as the preferred method for weed control, together with the lack of available sunflower selective herbicides makes the development of sunflower traits with resistance to herbicides crucial (Sala et al. 2012).

### SUMMARY

Experiments to develop a robust sunflower ER methodology were designed and carried out, with the objective of shortening the life cycle in our commercial Sunflower Trait Introgression (TI) process to ≤85 days, embryo to embryo. Application of this technology enables us to conduct 4 instead of 3 generations per year, enabling faster delivery of traited Sunflower hybrids to the market. In addition, we have established an in vitro herbicidal selection protocol as part of the ER process that improves the efficiency of our TI process by reducing population sizes of inbred lines being converted to a herbicide tolerance trait.

Presented here are methods and compositions as follows: 1) a method for harvesting sunflower immature embryos that enables efficient generation of plantlets from embryo rescue (ER); 2) a robust and genotype-independent method for in vitro ER; 3) a robust in vitro herbicidal selection methodology; and 4) plants generated by the methods described.

### BRIEF DESCRIPTION OF THE FIGURES

FIG. 1 shows the immature seed from outer row 2 to inner row 8 of flower head 14 days after pollination.

FIG. 2 shows immature embryos placed on medium after isolation.

FIG. 3 shows an example of a healthy ER plantlet with ≥1.5 cm shoot height from root crown to cotyledon.

FIG. 4 shows and example of healthy ER plants with 2-4 roots with lateral roots and true leaves.

### DEFINITIONS

As used herein, “AIR” refers to one of the mutations in the Ahasl1 gene that results in tolerance to an acetolactate synthase (AHAS) inhibiting herbicide. Additionally, “AIR” also refers to the trait providing resistance to said herbicides.

As used herein, “healthy ER plantlet growth” refers to plantlets having a shoot height (between cotyledon and root crown region) ≥1.5 cm with ≥85% of them having healthy roots.

As used herein, “healthy roots” refers to plants with 2-4 main roots as well as lateral roots.

As used herein, “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait.

As used herein, “desired trait, allele, or phenotype” refers to a characteristic of interest in the wild Helianthus species that is desired in the domestic Helianthus species. Such a “trait, allele, or phenotype” can include resistance to organisms causing broomrape or to diseases. Alternatively, such a trait, allele, or phenotype can include improved yield, protein content, oil content and composition, drought tolerance, and flowering times.

As used herein, “introgressed” refers to the introduction of a trait, allele, or phenotype from the genome of one plant, from e.g. a wild Helianthus plant, into the genome of another plant, e.g. domesticated Helianthus, that lacks such trait, allele, or phenotype.

As used herein, “chromosome” refers to, as recognized in the art, the self-replicating genetic structure in the cellular nucleus containing the cellular DNA and bearing the linear array of genes.

As used herein, “self” or “selfing” refers to the production of seed by self-fertilization or self-pollination, i.e. pollen and ovule are from the same plant.

As used herein, “F2” refers to the second filial generation.

As used herein, “dicot species” refers to plant species that are a part of the dicotyledon group in that the seed of the plant possesses two embryonic leaves (cotyledons).

As used herein, “plant” refers to any plant at any stage of development, particularly a seed plant.

As used herein, the term “plant part” refers to and indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like.

As used herein, the term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

As used herein, “herbicide” refers to a substance or compound that is toxic to and used to control unwanted plants (e.g. weeds). An herbicide can be selective (specific to species) or non-selective (broad).

As used herein, “herbicide tolerance” refers to a plant's ability to withstand herbicides and avoiding harm.

As used herein, “backcrossing” refers to a process in which a hybrid progeny is repeatedly crossed back to one of the parents.

### DETAILED DESCRIPTION

The current invention includes a method for a plant embryo rescue. Steps of the methods comprise a) harvesting and sterilizing immature seeds at 20 or fewer days after pollination (“DAP”); b) isolating immature embryos from the immature seeds of step a; c) culturing the immature embryos of step b; and d) growing plantlets from the cultured immature embryos of step c in suitable growth media and conditions. The suitable growth media of the method comprises a sucrose concentration, a photoperiod, and a temperature optimized for healthy ER plantlet growth.

The plant of the method above is a dicot species and in another embodiment is a sunflower. The immature embryo age for enhanced plantlet development in the method is 10 to 18 days after pollination. In one embodiment, the immature embryo age is 12 days after pollination. In another embodiment, the immature embryo age is 14 days after pollination. The embryo is in tissue culture for 5 to 12 days without sub-culture. In one embodiment, the embryo is in tissue culture for 7 days without sub-culturing. In another embodiment, the embryo is in tissue culture for 10 days with sub-culturing. The sucrose concentration in the embryo rescue method is 15 g/l to 30 g/l. In another embodiment, the sucrose concentration is 20 g/l. The photoperiod in the method for embryo growth is one of i) 16-hour day/8-hour night, ii) 8-hour day/16-hour night, iii) 5 day night plus 16-hour day/8-hour night for 2 days, or iv) 3 days night plus 16-hour day/8-hour night for 4 days. In one embodiment, the photoperiod for embryo growth is 3 days night plus 16-hour day/8-hour night for 4 days. In another embodiment, the photoperiod for embryo growth is 16-hour day/8-hour night. The temperature for embryo growth is 20° C. to 30° C. In another embodiment, the temperature is 25° C. The invention includes a plant, plant part, or progeny thereof produced by the embryo rescue method.

In another embodiment, the invention includes a method for in vitro herbicidal trait selection using the embryo rescue methodology previously described. The immature embryos are grown on a culture medium comprising an herbicide. The herbicide is selected from the group consisting of imidazolinones, pyrimidinylthiobenzoates, sulfonylaminocarbonyltriazolinone, sulfonylureas, triazolopyrimidines, amino acid derivatives, isoxazoles, pyrazolones, or triketones. The herbicide resistance gene is selected from the group consisting of Ahasl1, EPSPS, PAT, or an HPPD-inhibitor resistance gene. In one embodiment, the trait is the AIR herbicidal tolerance trait. The herbicide of the culture medium of the method comprises bensulfuron-methyl (BSM) or metsulfuron-methyl (MSM). The BSM concentration is 100 nM to 500 nM and in another embodiment, the BSM concentration is 300 nM. The MSM concentration is 1 nM to 250 nM and in another embodiment, the MSM concentration is 5 nM. In an embodiment, the immature embryos of the method are harvested 10 to 18 days post pollination. In one embodiment, the immature embryos are harvested 12 days post pollination. In another embodiment, the immature embryos are harvested 14 days post pollination. The immature embryos are in tissue culture for 7 to 10 days without sub-culturing. In one embodiment, the immature embryos are in tissue culture for 10 days without sub-culturing. The method further comprises growing selected plantlets into plants and backcrossing with another plant to obtain another generation. In an embodiment, the embryos are harvested from a dicot species and in another embodiment, the species is sunflower. Finally, the herbicidal trait selection method of the invention includes a plant, plant part, or progeny thereof, wherein the plant, plant part, or progeny thereof is produced by the method of the invention.

### EXAMPLES

### Example #. Standardization of Immature Embryo Extraction Methodology

For sunflower inbred line FS703RM1 immature embryo length ranged from 7.1 mm to 7.4 mm and 7.7 mm to 8.1 mm from row 1 to row 7, 10 and 12 days after pollination, respectively (Table 1). There was variation in leaf, shoot and root growth 7 days after tissue culture for immature embryos from rows 1-7 in the flower head, 10 and 12 days after pollination (Table 2). The conclusion was that immature embryo age needed to be determined by identifying the flowering sequence pattern for a given genotype, to enable the standardization of immature embryo ages and reduce the observed variation between embryos from different rows in the flower head (see Sunflower Embryo Rescue Protocol below for methodology on how to determine age and extract immature embryos).

TABLE 1
Immature embryo size of sunflower inbred line FS703RM1 from
row 1 to row 7, extracted 10 and 12 days after pollination.

| | Mean^{x} of immature | Mean^{x} of immature |
|---|---|---|
| | embryo length (mm) | embryo length (mm) |
| | extracted 10 days after | extracted 12 days after |
| Position of immature | pollination ± standard | pollination ± standard |
| embryos in flower head | deviation | deviation |
| Row 1 | 7.1 ± 0.3 | 7.7 ± 0.2 |
| Row 2 | 7.1 ± 0.4 | 8.0 ± 0.2 |
| Row 3 | 7.3 ± 0.4 | 8.1 ± 0.2 |
| Row 4 | 7.4 ± 0.3 | 7.9 ± 0.2 |
| Row 5 | 7.2 ± 0.3 | 8.0 ± 0.2 |
| Row 6 | 7.4 ± 0.2 | 7.8 ± 0.2 |
| Row 7 | 7.1 ± 0.2 | 7.9 ± 0.2 |

^{x}Ten immature embryos were measured per row

TABLE 2
Effect of immature embryos from row 1 to row 7, 10 and 12 days after
pollination on new leaf growth, shoot growth and root growth 7
days after tissue culture of sunflower inbred line FS703RM1.

| Position | | | Frequency (%) of | | Frequency (%) of | |
|---|---|---|---|---|---|---|
| of | Mean of shoot height of | | immature embryos | | immature embryos | |
| immature | immature embryos | | forming roots 7 d after | | forming new leaves 7 d | |
| embryos | (cm) 7 d after culture | | culture | | after culture | |
| in flower | 10 d after | 12 d after | 10 d after | 12 d after | 10 d after | 12 d after |
| head | pollination | pollination | pollination | pollination | pollination | pollination |
| Row 1 | 1.1 | 0.6 | 50.0 | 60.0 | 70.0 | 100.0 |
| Row 2 | 0.8 | 0.7 | 30.0 | 50.0 | 70.0 | 100.0 |
| Row 3 | 0.7 | 0.8 | 40.0 | 30.0 | 100.0 | 100.0 |
| Row 4 | 0.5 | 0.6 | 50.0 | 50.0 | 100.0 | 100.0 |
| Row 5 | 0.7 | 0.7 | 60.0 | 40.0 | 100.0 | 100.0 |
| Row 6 | 0.8 | 0.6 | 40.0 | 30.0 | 100.0 | 100.0 |
| Row 7 | 0.6 | 0.5 | 20.0 | 20.0 | 100.0 | 90.0 |

Ten immature embryos were measured per row.

### Example 1. Effects of Sucrose Concentration, Photoperiod, Temperature and Genotype on Immature Embryo Growth

Four parameters were measured and optimized for the generation of quality plantlets from tissue culture for embryo rescue (ER): i) percentage of immature embryos forming leaves; ii) percentage of immature embryos forming visible roots; iii) percentage of immature embryos forming healthy roots (2-4 main roots with lateral roots); and iv) shoot height as measured from the root crown region to the cotyledon. Healthy ER plantlets were defined as having a shoot height ≥1.5 cm and >85% of them having healthy roots and normal growth 10 days after transplanting to the greenhouse. To determine optimal growth conditions for ER-derived plantlet production, 18 different initial treatments were evaluated including:
three different sucrose concentrations (10, 30 and 60 g/l),
two different temperatures (25° C. and 30° C.),
three different photoperiods (16-hour day/8-hour night; 8-hour day/16-hour night; and 5 day night+16-hour day/8-hour night for 2 days),
two different embryo ages (10- and 12-days post-pollination), and
two genotypes (sunflower inbred lines FS703RM1 and SF564).

Leaf Development

For leaf development, there were only a few situations that resulted in an inhibitory effect: a photoperiod of 5 days night+16-hour day/8-hour night for 2 days at both temperatures (25° C. and 30° C.) for 10-day old immature embryos using sunflower inbred line FS703RM1.

Shoot Elongation

Shoot elongation was inhibited by 30 and 60 g/l of sucrose compared to 10 g/l under all photoperiod, temperature, and age conditions for both genotypes. Shoot elongation was enhanced under shorter light periods (8-hour day/16-hour night; and 5-day night+16-hour day/8-hour night for 2 days) compared with the 16-hour day/8-hour night photoperiod. Older (12 day old) immature embryos had increased shoot elongation compared to 10-day old immature embryos, under 10 g/l sucrose conditions for both genotypes.

Root Development

Twelve-day old immature embryos of line FS703RM1 exhibited enhanced root development compared to 10-day old immature embryos under most of the conditions tested. Root development was inhibited when tested with 60 g/l sucrose under a 16-hour day/8-hour night photoperiod at 25° C. Sucrose at 60 g/l had an inhibitory effect on root development compared with 10 and 30 g/l sucrose. For SF564, 12-day old immature embryos had less root development when using higher (30 and 60 g/l) sucrose concentrations, the one exception being for 30 g/l sucrose under the 5-day night+16-hour day/8-hour night for 2 days photoperiod at 30° C. for this genotype.

Summary

Summarizing, sucrose concentration, temperature, photoperiod and embryo age did not affect leaf development of ER plantlets for the sunflower inbred lines FS703RM1 and SF564. On the other hand, sucrose concentration and embryo age had important effects on both shoot elongation and root development of ER plantlets. Photoperiod affected shoot elongation specifically. However, further experiments were done to optimize these parameters.

From the above results, we decided to further evaluate the sucrose concentration, photoperiod, temperature and embryo age for immature embryo growth for both lines (FS703RM1 and SF564). The top five combinations of sucrose concentration, photoperiod, and temperature were selected from previous experimental results based on the shoot height and percentage of immature embryos forming roots or with healthy roots using 10 and 12-day old immature embryos for FS703RM1. The top four tissue culture condition combinations of sucrose concentration, photoperiod, and temperature were selected for FS564.

Sucrose at 20 g/l produced longer shoots (up to 1.1-fold longer) than those produced at 10 g/l sucrose across different embryo ages and genotypes (with the exception of 10 g/l sucrose under the 16-hour day/8-hour night photoperiod at 25° C. for genotype FS703RM1). See Tables 3 and 4 below. In addition, sucrose at 20 g/l generated a higher percentage (up to twice compared to 10 g/l) of immature embryos with healthy roots for both embryo ages and genotypes using line FS703RM1 under the 16-hour day/8-hour night photoperiod at 25° C. (Tables 3 and 4).

TABLE 3
Effect of sucrose concentration on shoot and root growth
of immature embryos for sunflower inbred line FS703RM1.

| | Mean of shoot height of | | Frequency of immature | |
|---|---|---|---|---|
| | immature embryos 7 days | | embryos forming healthy roots | |
| | after culture (cm) | | 7 days after culture (%) | |
| | 10 days | 12 days | 10 days | 12 days |
| | after | after | after | after |
| | polli- | polli- | polli- | polli- |
| Treatment | nation | nation | nation | nation |
| 10 g/l sucrose + | 1.3 | 1.7 | 80.0 | 100.0 |
| 16/8 + 25° C. | | | | |
| 20 g/l sucrose + | 1.4 | 1.6 | 90.0 | 95.0 |
| 16/8 + 25° C. | | | | |
| 10 g/l sucrose + | 1.9 | 2.5 | 70.0 | 85.0 |
| 5 dark + 25° C. | | | | |
| 20 g/l sucrose + | 1.9 | 2.1 | 75.0 | 90.0 |
| 5 dark + 25° C. | | | | |
| 10 g/l sucrose + | 1.5 | 1.3 | 65.0 | 90.0 |
| 8/16 + 25° C. | | | | |
| 20 g/l sucrose + | 1.2 | 1.3 | 79.0 | 95.0 |
| 8/16 + 25° C. | | | | |

Two to four experiments were conducted with 39 to 80 explants per treatment.

TABLE 4
Effect of sucrose concentration on shoot and root growth
of immature embryos for sunflower inbred line SF564.

| | Mean of shoot height of | | Frequency of immature | |
|---|---|---|---|---|
| | immature embryos 7 days | | embryos forming healthy roots | |
| | after culture (cm) | | 7 days after culture (%) | |
| | 10 days | 12 days | 10 days | 12 days |
| | after | after | after | after |
| | polli- | polli- | polli- | polli- |
| Treatment | nation | nation | nation | nation |
| 10 g/l sucrose + | 1.4 | 2.5 | 5.0 | 42.0 |
| 5 dark + 30° C. | | | | |
| 20 g/l sucrose + | 3.4 | 5.2 | 50.0 | 85.0 |
| 5 dark + 30° C. | | | | |
| 10 g/l sucrose + | 1.2 | 1.9 | 15.0 | 50.0 |
| 5 dark + 25° C. | | | | |
| 20 g/l sucrose + | 2.4 | 2.3 | 55.0 | 80.0 |
| 5 dark + 25° C. | | | | |
| 10 g/l sucrose + | 1.1 | 1.4 | 22.0 | 45.0 |
| 3 dark + 25° C. | | | | |
| 20 g/l sucrose + | 1.6 | 1.9 | 74.0 | 90.0 |
| 3 dark + 25° C. | | | | |
| 10 g/l sucrose + | 1.2 | 1.2 | 50.0 | 40.0 |
| 16/8 + 30° C. | | | | |
| 20 g/l sucrose + | 1.4 | 1.5 | 63.0 | 80.0 |
| 16/8 + 30° C. | | | | |
| 10 g/l sucrose + | 0.8 | 1.1 | 25.0 | 60.0 |
| 16/8 + 25° C. | | | | |
| 20 g/l sucrose + | 1.0 | 1.3 | 65.0 | 80.0 |
| 16/8 + 25° C. | | | | |
| 10 g/l sucrose + | 0.9 | 1.2 | 55.0 | 55.0 |
| 8/16 + 30° C. | | | | |
| 20 g/l sucrose + | 1.0 | 1.4 | 55.0 | 70.0 |
| 8/16 + 30° C. | | | | |
| 10 g/l sucrose + | 0.8 | 1.1 | 25.0 | 45.0 |
| 8/16 + 25° C. | | | | |
| 20 g/l sucrose + | 0.8 | 1.5 | 45.0 | 70.0 |
| 8/16 + 25° C. | | | | |

One experiment was conducted with 20 explants per treatment.

For FS703RM1, only one tissue culture condition (12-day old embryos on 20 g/l sucrose under the 16-hour day/8-hour night photoperiod at 25° C.) met our criteria (a shoot height at least 1.5 cm with at least 85% of them having healthy roots) for success. For FS564, three tissue culture conditions with 12-day old embryos and 20 g/l sucrose (25° C. and 30° C. under the 16-hour day/8-hour night photoperiod, and 30° C. under the 8-hour day/16-hour night photoperiod) met our criteria for success. The 10-day old immature embryos did not generate ER plantlets meeting our success criteria in any of the combinations. Consequently, we chose 12-day old immature embryos for subsequent experiments. See Tables 5, 6 and 7 below.

TABLE 5
Effect of combination of tissue culture conditions on shoot
and root growth of immature embryos 10 days and 12 days
after pollination for sunflower inbred line FS703RM1.

| | Mean of shoot height of | | Frequency of immature | |
|---|---|---|---|---|
| | immature embryos 7 days | | embryos forming healthy roots | |
| | after culture (cm) | | 7 days after culture (%) | |
| | 10 days | 12 days | 10 days | 12 days |
| | after | after | after | after |
| | polli- | polli- | polli- | polli- |
| Treatment | nation | nation | nation | nation |
| 10 g/l sucrose + | 1.3 | 1.6 | 80.0 | 82.5 |
| 16/8 + 25° C. | | | | |
| 10 g/l sucrose + | 1.0 | 1.1 | 75.0 | 65.0 |
| 8/16 + 25° C. | | | | |
| 10 g/l sucrose + | 2.1 | 2.4 | 71.7 | 80.0 |
| 5 dark + 25° C. | | | | |
| 20 g/l sucrose + | 1.3 | 1.7 | 81.8 | 97.5 |
| 16/8 + 25° C. | | | | |
| 10 g/l sucrose + | 2.3 | Not tested | 80.0 | Not tested |
| 8/16 + 30° C. | | | | |

Two to four experiments were conducted with 40 to 80 explants per treatment.

TABLE 6
Effect of combination of tissue culture conditions
on shoot and root growth of immature embryos 10 days
after pollination for sunflower inbred line FS564.

| | Mean of shoot | Frequency of |
|---|---|---|
| | height of immature | immature embryos |
| | embryos 7 days | forming healthy roots |
| Treatment | after culture (cm) | 7 days after culture (%) |
| 20 g/l sucrose + | 2.3 | 63.9 |
| 5 dark + 25° C. | | |
| 10 g/l sucrose + | 1.4 | 66.2 |
| 16/8 + 30° C. | | |
| 20 g/l sucrose + | 1.4 | 61.0 |
| 16/8 + 30° C. | | |
| 20 g/l sucrose + | 1.5 | 65.8 |
| 16/8 + 25° C. | | |

Two to four experiments were conducted with 40 to 80 explants per treatment.

TABLE 7
Effect of combination of tissue culture conditions
on shoot and root growth of immature embryos 12 days
after pollination for sunflower inbred line FS564.

| | Mean of shoot | Frequency of |
|---|---|---|
| | height of immature | immature embryos |
| | embryos 7 days | forming healthy roots |
| Treatment | after culture (cm) | 7 days after culture (%) |
| 20 g/l sucrose + | 1.8 | 87.1 |
| 16/8 + 30° C. | | |
| 20 g/l sucrose + | 1.6 | 87.5 |
| 16/8 + 25° C. | | |
| 20 g/l sucrose + | 1.6 | 85.0 |
| 8/16 + 30° C. | | |
| 20 g/l sucrose + | 1.5 | 81.4 |
| 8/16 + 25° C. | | |

Two to four experiments were conducted with 40 to 80 explants per treatment.

### Example 2. Evaluation of ER Methodology in Additional Cytoplasmic Male Sterile (CMS) Genotypes

We evaluated the ER methodology on a broader diversity of genotypes. The top tissue culture conditions described from each of FS703RM1 and FS564 were tested in additional lines using 10 and 12 day old immature embryos. The top tissue culture conditions tested were: i) 20 g/l sucrose with a 16-hour day/8-hour night photoperiod at 25° C.; ii) 20 g/l sucrose with a 16-hour day/8-hour night photoperiod at 30° C.; iii) 10 g/l sucrose with a 16-hour day/8-hour night photoperiod at 25° C.; iv) 20 g/l sucrose with a 8-hour day/16-hour night photoperiod at 25° C.; and v) 20 g/l sucrose with a 5 days dark+16-hour day/8-hour night for 4 days photoperiod at 25° C.

For line AS10277, four tissue culture conditions generated ER plantlets that met our success criteria for healthy ER plantlets (shoot height ≥1.5 cm and >85% with healthy roots) with 12-day old immature embryos. For line FS73100, only one combination culture condition with 12-day old immature embryos (20 g/l sucrose with a 3 days dark+16-hour day/8-hour night for 4 days photoperiod at 25° C.) produced ER plantlets that met our success criteria. For lines FS75400, AD4071 and AE78079, none of the tested tissue culture conditions generated ER plantlets that met our success criteria although they produced healthy ER plantlets at frequencies up to 76%, 83% and 80%. See Table 8 below for the corresponding above data.

TABLE 8
Effect of top five combinations of tissue culture conditions
on shoot and root growth of immature embryos 10 days
and 12 days after pollination for sunflower inbred lines
AS10277, FS73100, FS75400, AD4071 and AE78079.

| | | Mean shoot height | | Frequency of embryos | |
|---|---|---|---|---|---|
| | | 7 d after | | forming health roots | |
| | | culture (cm) | | 7 d after culture (%) | |
| | | 10 d | 12 d | 10 d | 12 d |
| | | after | after | after | after |
| | | polli- | polli- | polli- | polli- |
| Treatment | Genotype | nation | nation | nation | nation |
| 20S+ 16/8 + | AS10277 | 0.8 | 1.7 | 49. | 88 |
| 25° C. | | | | | |
| 10S+16/8 + | | 0.6 | 1.9 | 50 | 88 |
| 25° C. | | | | | |
| 20S +8/16 + | | 0.8 | 1.8 | 47 | 93 |
| 25° C. | | | | | |
| 20S + 16/8 + | | 1.0 | 2.1 | 44 | 85 |
| 30° C. | | | | | |
| 20S + 5 dark + | | 0.9 | 2.3 | 26 | 77 |
| 16/8 + 25° C. | | | | | |
| 20S + 16/8 + | FS73100 | 2.0 | 2.3 | 73 | 80 |
| 25° C. | | | | | |
| 10S+ 16/8 + | | 2.0 | 2.5 | 73 | 82 |
| 25° C. | | | | | |
| 20S+8/16 + | | 2.0 | 2.4 | 79 | 82 |
| 25° C. | | | | | |
| 20S + 16/8 + | | 2.1 | 2.9 | 63 | 70 |
| 30° C. | | | | | |
| 20S + 5 dark + | | 2.3 | 1.9 | 60 | 85 |
| 16/8 + 25° C. | | | | | |
| 20S + 16/8 + | FS75400 | 1.5 | 1.9 | 39 | 76 |
| 25° C. | | | | | |
| 10S+ 16/8 + | | 1.6 | 1.9 | 38 | 70 |
| 25° C. | | | | | |
| 20S+8/16 + | | 1.5 | 2.3 | 34 | 61 |
| 25° C. | | | | | |
| 20S +16/8 + | | 1.6 | 2.8 | 21 | 58 |
| 30° C. | | | | | |
| 20S + 5 dark + | | 1.7 | 2.4 | 24 | 54 |
| 16/8 + 25° C. | | | | | |
| 20S +16/8 + | AD40713 | 0.9 | 1.6 | 37 | 83 |
| 25° C. | | | | | |
| 10S +16/8 + | | 0.9 | 1.7 | 25 | 73 |
| 25° C. | | | | | |
| 20S+8/16 + | | 1.0 | 2.0 | 28 | 82 |
| 25° C. | | | | | |
| 20S+ 16/8 + | | 0.8 | 2.0 | 27 | 58 |
| 30° C. | | | | | |
| 20S + 5 dark + | | 1.3 | 1.9 | 45 | 82 |
| 16/8 + 25° C. | | | | | |
| 20S +16/8 + | AE78079 | 1.5 | 1.2 | 78 | 75 |
| 25° C. | | | | | |
| 10S+16/8 + | | 1.5 | 1.4 | 70 | 50 |
| 25° C. | | | | | |
| 20S+8/16 + | | 1.8 | 1.7 | 80 | 73 |
| 25° C. | | | | | |
| 20S + 16/8 + | | 1.7 | 1.6 | 58 | 75 |
| 30° C. | | | | | |
| 20S + 5 dark + | | 1.9 | 1.6 | 53 | 38 |
| 16/8 + 25° C. | | | | | |

“20S” represents 20 g/l sucrose, and “10S” represents 10 g/l sucrose.
Three to six experiments were conducted with 60 to 120 explants per treatment.

### Example 3. Effects of Immature Embryo Age on Enhancing ER Plantlet Development

In experiments using the tissue culture conditions described above with 10 and 12 day old immature embryos, three of the five sunflower inbred lines did not meet the success criteria for ER plantlets. In addition, among four sunflower inbred lines generating ER plantlets that did meet the defined success criteria, three sunflower inbred lines (FS703RM1, FS564, AS10277) generated healthy ER plantlets under the same conditions: 20 g/l sucrose under a 16-hour day/8-hour night photoperiod at 25° C. The other sunflower inbred line (FS73100) produced healthy ER plantlets under different conditions (20 g/l sucrose with a 3 days dark+16-hour day/8-hour night for 4 days photoperiod at 25° C.).

For trait introgression pipeline production, a single culture condition across different lines is desirable. Therefore, the condition (20 g/l sucrose with a 16-hour day/8-hour night photoperiod at 25° C.) was selected to examine the effect of additional immature embryo ages (14, 16 and 18 days post-pollination) on enhancing ER plantlet development for the 5 sunflower inbred lines that previously failed to meet the defined success criteria.

Sunflower inbred lines AE78079, FS73100, A531, FS75400 and AD40713 had 88.3%, 98.4%, 87.8%, 95%, and 93.3% to 98.9% of immature embryos producing healthy ER plantlets when using immature embryos 14 days or 16 days after pollination, respectively (Table 9). All eight sunflower inbred lines tested were able to generate healthy ER plantlets at rates meeting the success criteria, ranging from 87.5% to 98.4% under a single 7-day tissue culture regime using immature embryos 12, 14 or 16 days after pollination. In addition, there were no statistically significant differences in success rates in generating healthy ER plantlets between the eight sunflower inbred lines tested (Table 10), indicating that this ER methodology is genotype independent. Using 14 or 16-day old immature embryos overcame the genotype-dependency observed when using 12-day old immature embryos.

TABLE 9
Frequency of immature embryos of different ages forming healthy shoots
and healthy shoots for sunflower inbred lines AE78079, FS73100,
FS75400, A531 and AD40713 using one culture condition of sucrose
20 g/l at 25° C. under 16-hour day/8-hour night photoperiod.

| | | Mean of shoot | Frequency of |
|---|---|---|---|
| | | height of immature | immature embryos |
| Age at | | embryos 7 days | forming healthy roots |
| Treatment | Genotype | after culture (cm) | 7 days after culture (%) |
| 12 days | AE78079 | 1.4 | 81.7 |
| 14 days | | 1.9 | 88.3 |
| 16 days | | 1.4 | 65.0 |
| 18 days | | 0 | 0.0 |
| 12 days | FS73100 | 1.8 | 85.0 |
| 14 days | | 3.1 | 98.4 |
| 16 days | | 2.7 | 75.0 |
| 18 days | | 1.8 | 63.3 |
| 12 days | FS75400 | 2.0 | 74.2 |
| 14 days | | 2.4 | 84.2 |
| 16 days | | 3.5 | 95.0 |
| 18 days | | 0.6 | 50.5 |
| 12 days | A531 | 1.6 | 57.8 |
| 14 days | | 3.0 | 87.8 |
| 16 days | | 1.0 | 34.4 |
| 18 days | | 0.2 | 4.4 |
| 12 days | AD40713 | 1.5 | 83.3 |
| 14 days | | 2.2 | 93.3 |
| 16 days | | 3.1 | 98.9 |
| 18 days | | 0.1 | 4.4 |

Two to four experiments were conducted with 60 to 120 explants per treatment.

TABLE 10
Frequency of immature embryos forming healthy
shoots and healthy plantlets at the optimal
embryo age for each of 8 inbred lines tested.

| | | Mean of shoot | Frequency^{x} of |
|---|---|---|---|
| | | height of immature | immature embryos |
| | | embryos 7 days | forming healthy plantlets |
| Treatment | Genotype | after culture (cm) | 7 days after culture (%) |
| 12 days | FS703RM1 | 1.6 | 97.5 A |
| after | SF564 | 1.6 | 87.5 A |
| pollination | AS10277 | 1.7 | 88.3 A |
| 14 days | FS73100 | 3.1 | 98.4 A |
| after | AE78079 | 1.9 | 88.3 A |
| pollination | A531 | 3.0 | 87.8 A |
| | AD40713 | 2.2 | 93.3 A |
| 16 days | FS75400 | 3.5 | 95.0 A |
| after | | | |
| pollination | | | |

^{x}Numbers with the same letter in each bar are not statistically significantly (P > 0.05) different.
Two to four experiments were conducted with 60 to 120 explants per treatment.

### Example 4. Seed Yield of Plants Derived from ER Tissue Culture Under Greenhouse Conditions

To evaluate potential effects on plants derived from ER tissue culture, 5 ER-derived and 5 seed-grown (control) plants for each of the sunflower inbred lines previously tested were grown to maturity in parallel. Plants were grown in the greenhouse and the yields compared. No statistically significant differences in yield performance were observed between ER-derived and seed-grown plants for these lines (Table 11), indicating that the ER methodology does not negatively affect seed yield.

The ER methodology can be defined as simple (simple explant and medium, easy embryo extraction), rapid (7 day single tissue culture cycle, no sub-culturing), high throughput, genotype-independent (87.8% to 98.9% of immature embryos producing healthy ER plantlets survived in the greenhouse across eight different sunflower inbred lines tested), and producer of high quality ER plantlets (i.e. with the same seed yield as seed-grown plants; see Table 11).

TABLE 11
Average seed yields for ER-derived and seed-grown
plants as control (CK) grown in the greenhouse
for different sunflower inbred lines tested.

| | | Average yield^{x} |
|---|---|---|
| Genotype | Treatment | per plant (g) |
| AS10277 | CK plant | 42.9 A |
| | ER plant/12 days | 46.4 A |
| FS703RM1 | CK plant | 46.9 B |
| | ER plant/12 days | 50.1 B |
| SF564 | CK plant | 70.3 C |
| | ER plant/12 days | 78.0 C |
| FS73100 | CK plant | 64.4 D |
| | ER plant/14 days | 68.0 D |
| FS75400 | CK plant | 87.5 E |
| | ER plant/14 days | 92.3 E |
| A531 | CK plant | 53.1 F |
| | ER plant/14 days | 57.2 F |
| AE78079 | CK plant | 21.7 G |
| | ER plant/14 days | 34.1 G |

^{x}Numbers with the same letter are not statistically significantly (P > 0.05) different.
Average yield from at least five plants for each treatment.

### Example 5. Minimal In Vitro Culture Period to Identify Immature Embryos Bearing the Herbicidal Tolerance Trait

To determine the minimal in vitro tissue culture period needed to adequately segregate the AIR herbicidal tolerance trait, 12 day old immature embryos of the first backcross generation (BC1) of an AIR_3U trait conversion were cultured for 7 and 10 days in SF germ 5 media (see Example 12 below). The media was supplemented with bensulfuron-methyl (BSM) at 100 and 500 nM and metsulfuron-methyl (MSM) at 10 nM.

The segregation ratios of trait-positive (forming roots) and trait-negative (not forming roots) plantlets based on visual scoring were noted and analyzed by in a chi-square statistical test. For both herbicides, at the different concentrations tested, trait-positive and trait-negative plantlets derived from immature embryos cultured for 10 days segregated as predicted (P>0.05, Table 12). However, trait-positive and trait-negative plantlets derived from 7 days after tissue culture did not (P<0.05, Table 12). Therefore, a 10 day in vitro tissue culture selection period was chosen for both herbicides to ensure that visual scoring matched the expected segregation ratio for the herbicidal selection trait.

TABLE 12
AIR trait segregation for back-cross generation 1 (BC1)
AIR RD506011KMZ (AIR_3U) using bensulfuron-methyl (BSM)
metsulfuron-methyl (MSM) 7 and 10 days after culture.

| | Air | Observed | Expected | | |
|---|---|---|---|---|---|
| Treatment | Trait | No. | No. | χ² | P value |
| 10 nM MSM | Positive | 17 | 29.25 | | |
| 7 days | Negative | 22 | 9.75 | | |
| | Total | 39 | 39 | 20.521 | <0.05 |
| 10 nM MSM | Positive | 24 | 29.25 | | |
| 10 days | Negative | 15 | 9.75 | | |
| | Total | 39 | 39 | 3.769 | >0.05 |
| 100 nM BSM | Positive | 22 | 29.25 | | |
| 7 days | Negative | 17 | 9.75 | | |
| | Total | 39 | 39 | 7.188 | <0.05 |
| 100 nM BSM | Positive | 29 | 29.25 | | |
| 10 days | Negative | 10 | 9.75 | | |
| | Total | 39 | 39 | 0.009 | >0.05 |
| 500 nM BSM | Positive | 19 | 29.25 | | |
| 7 days | Negative | 20 | 9.75 | | |
| | Total | 39 | 39 | 14.368 | <0.05 |
| 500 nM BSM | Positive | 24 | 29.25 | | |
| 10 days | Negative | 15 | 9.75 | | |
| | Total | 39 | 39 | 3.769 | >0.05 |

P values are larger than 0.05; the observed ratios fit a 3:1 ratio.

### Example 6. Kill Curves and Visual Identification of Trait-Positive Plantlets in In Vitro Herbicidal Selection

We evaluated three parameters for visual screening of trait-positive plantlets: i) percentage of immature embryos producing new growth (including leaves and epicotyl elongation); ii) percentage of immature embryos producing small roots (1-2 tiny roots less than 0.5 cm in length); and iii) percentage of immature embryos producing healthy roots (2-4 main roots with lateral roots). To identify optimal herbicide concentrations for selection, initial experiments were performed using BSM at 500, 1000, and 5000 nM and MSM at 10, 50 and 250 nM on immature embryos harvested 12 days after pollination. Results revealed all the concentrations tested were too high, which resulted in the death of all immature embryos after 10 days in tissue culture (data not shown). Therefore, further experiments were conducted using BSM at 100, 300, and 500 nM and MSM at 1, 5 and 10 nM on immature embryos harvested 12 days after pollination of six BC1 AIR genotypes (wild-type absent of the herbicidal tolerance trait (confirmed by TaqMan analysis).

Across all six genotypes, none of the immature embryos formed healthy roots at the lowest concentration of 100 nM using BSM for selection. Ninety percent of AIR_67 embryos produced new growth but none of them formed small roots at 500 nM (Table 13). AIR_3T had 40% of embryos produce new growth and 23% of them formed small roots at 500 nM (Table 14). Only 4% of AIR_3W embryos produced new growth while 12% formed small roots at 500 nM (Table 15). AIR_3U had 98% of embryos produce new growth with 94% forming small roots at 500 nM (Table 16). AIR_3V had 22% of embryos produce new growth and form small roots at 500 nM (Table 17) Seventy-three percent of AIR_3X embryos produced new growth with 45% forming small roots at 500 nM (Table 18).

TABLE 13
Effect of bensulfuron-methyl (BSM) and metsulfuron-methyl
(MSM) on the growth of immature embryos (IE) 10 days after
culture for FT11244ZB (AIR_67, wild-type).

| | | % of IEs | % of IEs | % of IEs |
|---|---|---|---|---|
| | | forming | forming | forming |
| | Concentration | new | small | healthy |
| Herbicide | (nM) | growth | roots | roots |
| BSM | 0 | 100 | 0 | 95 |
| | 100 | 85 | 50 | 0 |
| | 300 | 85 | 10 | 0 |
| | 500 | 90 | 0 | 0 |
| MSM | 0 | 100 | 0 | 95 |
| | 1 | 80 | 85 | 0 |
| | 5 | 40 | 20 | 0 |
| | 10 | 0 | 0 | 0 |

Note:
three experiments were conducted with 30 explants per treatment.

TABLE 14
Effect of bensulfuron-methyl (BSM) and metsulfuron-methyl
(MSM) on the growth of immature embryos (IE) 10 days after
culture for RW666P3AIR (AIR_3T, wild-type).

| | | % of IEs | % of IEs | % of IEs |
|---|---|---|---|---|
| | Concentration | forming new | forming small | forming |
| Herbicide | (nM) | growth | roots | healthy roots |
| BSM | 0 | 100 | 2 | 85 |
| | 100 | 66 | 43 | 0 |
| | 300 | 41 | 26 | 0 |
| | 500 | 40 | 23 | 0 |
| MSM | 0 | 100 | 2 | 85 |
| | 1 | 100 | 75 | 0 |
| | 5 | 96 | 9 | 0 |
| | 10 | 74 | 0 | 0 |

Note:
five experiments were conducted with 51 explants per treatment.

TABLE 15
Effect of bensulfuron-methyl (BSM) and metsulfuron-methyl
(MSM) on the growth of immature embryos (IE) 10 days after
culture for FT11183ZB (AIR_3W, wild-type).

| | | % of IEs | % of IEs | % of IEs |
|---|---|---|---|---|
| | Concentration | forming new | forming small | forming |
| Herbicide | (nM) | growth | roots | healthy roots |
| BSM | 0 | 100 | 18 | 46 |
| | 100 | 20 | 38 | 0 |
| | 300 | 4 | 14 | 0 |
| | 500 | 4 | 12 | 0 |
| MSM | 0 | 100 | 18 | 46 |
| | 1 | 96 | 38 | 2 |
| | 5 | 76 | 8 | 0 |
| | 10 | 42 | 0 | 0 |

Note:
three experiments were conducted with 50 explants per treatment.

TABLE 16
Effect of bensulfuron-methyl (BSM) and metsulfuron-methyl
(MSM) on the growth of immature embryos (IE) 10 days
after culture for RD506011KMZ (AIR 3U, wild-type).

| | | % of IEs | % of IEs | % of IEs |
|---|---|---|---|---|
| | Concentration | forming new | forming small | forming |
| Herbicide | (nM) | growth | roots | healthy roots |
| BSM | 0 | 99 | 25 | 61 |
| | 100 | 98 | 98 | 0 |
| | 300 | 92 | 94 | 0 |
| | 500 | 98 | 94 | 0 |
| MSM | 0 | 99 | 25 | 61 |
| | 1 | 100 | 61 | 0 |
| | 5 | 18 | 2 | 0 |
| | 10 | 0 | 0 | 0 |

Note:
six experiments were conducted with 62 explants per treatment.

TABLE 17
Effect of bensulfuron-methyl (BSM) and metsulfuron-methyl
(MSM) on the growth of immature embryos (IE) 10 days after
culture for RT13187Z (AIR_3V, wild-type).

| | | % of IEs | % of IEs | % of IEs |
|---|---|---|---|---|
| | Concentration | forming new | forming small | forming |
| Herbicide | (nM) | growth | roots | healthy roots |
| BSM | 0 | 100 | 19 | 70 |
| | 100 | 57 | 73 | 0 |
| | 300 | 32 | 49 | 0 |
| | 500 | 22 | 30 | 0 |
| MSM | 0 | 100 | 19 | 70 |
| | 1 | 100 | 86 | 0 |
| | 5 | 100 | 14 | 0 |
| | 10 | 81 | 5 | 0 |

Note:
three experiments were conducted with 37 explants per treatment.

TABLE 18
Effect of bensulfuron-methyl (BSM) and metsulfuron-methyl
(MSM) on the growth of immature embryos (IE) 10 days after culture
for 19ALL111E3X MM (AIR_3X, wild-type).

| | | % of IEs | % of IEs | % of IEs |
|---|---|---|---|---|
| | Concentration | forming new | forming small | forming |
| Herbicide | (nM) | growth | roots | healthy roots |
| BSM | 0 | 100 | 8 | 75 |
| | 100 | 90 | 90 | 0 |
| | 300 | 83 | 65 | 0 |
| | 500 | 71 | 45 | 0 |
| MSM | 0 | 100 | 8 | 75 |
| | 1 | 100 | 65 | 0 |
| | 5 | 100 | 40 | 0 |
| | 10 | 53 | 0 | 0 |

Note:
two experiments were conducted with 40 explants per treatment.

Similarly, with one exception, for MSM at the lowest concentration of 1 nM, none of immature embryos from these genotypes formed healthy roots. The exception was AIR_3W, where 2% of the immature embryos formed healthy roots. At 10 nM MSM, the genotypes produced new growth ranging from 0 to 81%. None of the AIR_67 or AIR_3U embryos produced new growth. Seventy four percent of AIR_3T embryos produced new growth while 42% of AIR_3W produced new growth. AIR_3V and AIR_3X immature embryos had 81% and 53%, respectively, produce new growth. We observed 20%, 9%, 8%, 2%, 14% and 40% small root formation for AIR_67, AIR_3T, AIR_3W, AIR_3U, AIR_3V and AIR_3X immature embryos, respectively, at 5 nM. Respectively, we observed 85%, 75%, 38%, 61%, 86% and 65% small root formation at 1 nM MSM (Tables 13-18).

Ultimately, these results suggested that using new growth and small root observational parameters were ineffective in identifying trait-positive plantlets. High percentages of immature embryos exhibited new growth and small root formation even on media with high and medium concentrations of either herbicide for the wild-type genotypes tested. In contrast the healthy root formation observational parameter was an effective visual selection marker for tolerance to both herbicides because no immature embryos formed healthy roots at the lowest concentrations of 100 nM BSM and 1 nM MSM (except for AIR_3W with 2%) while still producing new shoot growth. These results also indicated that the concentrations of 100 nM BSM and 1 nM MSM were good starting points for determining optimal herbicide concentrations for in vitro selection of trait-positive plantlets.

### Example 7. Optimal Herbicide Concentrations for In Vitro Selection

Using the data from example 6, we evaluated the optimal herbicide concentrations for in vitro selection of trait-positive plantlets. Further examined were 1 and 5 nM MSM and 100 and 300 nM BSM across five BC1 AIR genotypes. Trait positive plants were selfed to generate F2 populations. Immature embryos were harvested from the F2 populations 12 days after pollination.

At 1 and 5 nM MSM, AIR_67 produced 30 tolerant plantlets for each. At 1 nM, 27 out of the 30 plantlets were confirmed as trait-positive by TaqMan analysis, and 30 out of 30 were trait-positive at the 5 nM concentration (Table 19). The other four AIR genotypes also generated plantlets tolerant to MSM at 5 nM and were all confirmed herbicide trait positive by TaqMan (Tables, 20, 21, 22, and 23). Based on these results, we determined that 5 nM MSM was an optimal concentration for in vitro selection. None of the AIR genotypes produced “escapes”, plantlets apparently herbicide tolerant that were in fact trait negative by TaqMan, at this concentration. AIR_67 generated 10% of “escapes” at 1 nM MSM.

Using BSM, AIR_3U generated 10 tolerant plantlets to BSM at 100 nM, but only 7 out of these 10 were herbicide trait-positive confirmed by TaqMan analysis (Table 20). The other four AIR genotypes tested produced plantlets tolerant to BSM at 300 nM and all of the plantlets were confirmed trait positive by TaqMan analysis (Tables, 19, 20, 21, 22, 23). These results indicated that 300 nM BSM was an optimal concentration for in vitro selection because at 100 nM BSM, AIR_3U generated 30% escapes.

TABLE 19
TaqMan analysis of FT11244ZB (AIR_67) herbicide tolerant
plants from in vitro selection with metsulfuron-methyl
(MSM) and bensulfuron-methyl (BSM) 10 days after culture
using immature embryos 12 days after pollination.

| | | No. of | |
|---|---|---|---|
| | | herbicide | |
| | No. of | resistant plants | % of herbicide |
| | herbicide | confirmedpositive | resistance plants |
| | resistant | by TaqMan | confirmed by |
| Treatment | plants tested | analysis | TaqMan analysis |
| 1 nM MSM | 30 | 27 | 90% |
| 5 nM MSM | 30 | 30 | 100% |
| 100 nM BSM | 30 | 30 | 100% |
| 300 nM BSM | 30 | 30 | 100% |

TABLE 20
TaqMan analysis of RD506011KMZ (AIR_3U) herbicide tolerant
plants from in vitro selection with metsulfuron-methyl
(MSM) and bensulfuron-methyl (BSM), 10 days after culture
using immature embryos 12 days after pollination.

| | | No. of | |
|---|---|---|---|
| | | herbicide | |
| | No. of | resistant plants | % of herbicide |
| | herbicide | confirmedpositive | resistance plants |
| | resistant | by TaqMan | confirmed by |
| Treatment | plants tested | analysis | TaqMan analysis |
| 1 nM MSM | 8 | 8 | 100% |
| 5 nM MSM | 7 | 7 | 100% |
| 100 nM BSM | 10 | 7 | 70% |
| 300 nM BSM | 5 | 5 | 100% |

TABLE 21
TaqMan analysis of RW666P3AIR (AIR_3T) herbicide tolerant
plants from in vitro selection with metsulfuron-methyl
(MSM) and bensulfuron-methyl (BSM), 10 days after culture
using immature embryos 12 days after pollination.

| | | No. of | |
|---|---|---|---|
| | | herbicide | |
| | No. of | resistant plants | % of herbicide |
| | herbicide | confirmedpositive | resistance plants |
| | resistant | by TaqMan | confirmed by |
| Treatment | plants tested | analysis | TaqMan analysis |
| 1 nM MSM | 30 | 30 | 100% |
| 5 nM MSM | 30 | 30 | 100% |
| 100 nM BSM | 30 | 30 | 100% |
| 300 nM BSM | 30 | 30 | 100% |

TABLE 22
TaqMan analysis of FT11183ZB (AIR_3W) herbicide tolerant
plants from in vitro selection with metsulfuron-methyl
(MSM) and bensulfuron-methyl (BSM), 10 days after culture
using immature embryos 12 days after pollination.

| | | No. of | |
|---|---|---|---|
| | | herbicide | |
| | No. of | resistant plants | % of herbicide |
| | herbicide | confirmedpositive | resistance plants |
| | resistant | by TaqMan | confirmed by |
| Treatment | plants tested | analysis | TaqMan analysis |
| 1 nM MSM | 30 | 30 | 100% |
| 5 nM MSM | 35 | 35 | 100% |
| 100 nM BSM | 30 | 30 | 100% |
| 300 nM BSM | 30 | 30 | 100% |

TABLE 23
TaqMan analysis of RT13187Z (AIR_3V) herbicide tolerant
plants from in vitro selection with metsulfuron-methyl
(MSM) and bensulfuron-methyl (BSM), 10 days after culture
using immature embryos 12 days after pollination.

| | | No. of | |
|---|---|---|---|
| | | herbicide | |
| | No. of | resistant plants | % of herbicide |
| | herbicide | confirmedpositive | resistance plants |
| | resistant | by TaqMan | confirmed by |
| Treatment | plants tested | analysis | TaqMan analysis |
| 5 nM MSM | 26 | 26 | 100% |

### Example 8. Growth of 14 and 16 Day Post Pollination Immature Embryos to Improve Identification of Herbicide Tolerant Trait-Positive Plantlets

Segregation of 12 day old trait-positive AIR_3W immature embryos on 300 nM BSM appeared abnormal—17.8% of trait-positive plantlets failed to grow or produce healthy roots (Table 24). However, we observed that 46% of immature embryos generated healthy roots on medium without BSM (Table 15) so the conclusion was that herbicide tolerant trait-positive plantlets were not able to produce healthy roots under selection. Therefore, older immature embryos (14 and 16 days post-pollination) were tested with the objective of improving the growth for three AIR BC1 genotypes (AIR_3W, AIR_3U, AIR_3V) that exhibited abnormal segregation for the herbicide tolerance trait when using 12 day old immature embryos. For AIR_3W, when using 14 day old immature embryos, the percentage of dead or abnormal trait-positive plantlets was reduced from 17.8% with 12 day old immature embryos and 16.7% with 16 day old immature embryos to 5% at 300 nM BSM (Table 24). Also, 14 day old immature embryos selected on 300 nM BSM and 5 nM MSM had, respectively, 100% and 98% trait-positive plants by TaqMan analysis as compared to 98% and 94%, respectively, for 16 day old immature embryos (Table 25).

TABLE 24
Herbicide trait segregation for BC1 AIR, FT11183ZB
(AIR_3W) using bensulfuron-methyl (BSM), 10 days after
culture using immature embryos 12, 14 and 16 days after pollination.

| | | | | % losing trait |
|---|---|---|---|---|
| | | | | positive |
| | | Observed | Expected | immature |
| Treatment | AIR trait | No. | No. | embryos |
| 300 nM BSM | Resistant | 37 | 45 | 17.8% |
| 12-day | | (out of 60) | | |
| embryos | | | | |
| 300 nM BSM | Resistant | 57 | 60 | 5.0% |
| 14-day | | (out of 80) | | |
| embryos | | | | |
| 300 nM BSM | Resistant | 50 | 60 | 16.7% |
| 16-day | | (out of 80) | | |
| embryos | | | | |

TABLE 25
TaqMan analysis of FT11183ZB (AIR_3W) herbicide tolerant
plants from in vitro selection using metsulfuron-methyl
(MSM) and bensulfuron-methyl (BSM), 10 days after culture
using immature embryos 14 and 16 days after pollination.

| | | No. of | % of |
|---|---|---|---|
| | No. of | herbicide | herbicide |
| | herbicide | tolerant plants | tolerant plants |
| | tolerant | confirmed positive | confirmed positive |
| Treatment | plants tested | by TaqMan | by TaqMan |
| 5 nM MSM; | 50 | 49 | 98% |
| 14-day | | | |
| embryos | | | |
| 5 nM MSM; | 53 | 50 | 94% |
| 16-day | | | |
| embryos | | | |
| 300 nM BSM; | 50 | 50 | 100% |
| 14-day | | | |
| embryos | | | |
| 300 nM BSM; | 49 | 48 | 98% |
| 16-day | | | |
| embryos | | | |

AIR_3V 14 day old immature embryos selected on 300 nM BSM and 5 nM MSM had, respectively, 100% and 98% trait-positive plants by TaqMan analysis compared to 93% and 90%, respectively, for 16 day old immature embryos (Table 26). For AIR_3U, 14 day old immature embryos selected on 300 nM BSM and 5 nM MSM had, respectively, 100% and 98% trait-positive plants by TaqMan analysis compared to 100% and 97%, respectively, of trait-positive plants for 16 day immature embryos (Table 27). These results suggest that 14 day old immature embryos were the optimal age for herbicidal selection for these three genotypes.

TABLE 26
TaqMan analysis of RT13187Z (AIR_3V) herbicide tolerant
plants from in vitro selection for using metsulfuron-methyl
(MSM) and bensulfuron-methyl (BSM), 10 days after culture
using immature embryos 14 and 16 days after pollination.

| | No. of | No. of herbicide | % of herbicide |
|---|---|---|---|
| | herbicide | tolerant plants | tolerant plants |
| | tolerant | confirmed positive | confirmed positive |
| Treatment | plants tested | by TaqMan | by TaqMan |
| 5 nM MSM; | 45 | 44 | 98% |
| 14-day | | | |
| embryos | | | |
| 5 nM MSM; | 51 | 46 | 90% |
| 16-day | | | |
| embryos | | | |
| 300 nM BSM; | 49 | 49 | 100% |
| 14-day | | | |
| embryos | | | |
| 300 nM BSM; | 44 | 41 | 93% |
| 16-day | | | |
| embryos | | | |

TABLE 27
TaqMan analysis of RD506011KMZ (AIR_3U) herbicide tolerant
plants from in vitro selection using metsulfuron-methyl
(MSM) and bensulfuron-methyl (BSM), 10 days after culture
using immature embyros 14 and 16 days after pollination.

| | | No. of | % of |
|---|---|---|---|
| | No. of | herbicide | herbicide |
| | herbicide | tolerant plants | tolerant plants |
| | tolerant | confirmed positive | confirmed positive |
| Treatment | plants tested | by TaqMan | by TaqMan |
| 5 nM MSM; | 44 | 44 | 100% |
| 14-day | | | |
| embryos | | | |
| 5 nM MSM; | 31 | 30 | 97% |
| 16-day | | | |
| embryos | | | |
| 300 nM BSM; | 38 | 38 | 100% |
| 14-day | | | |
| embryos | | | |
| 300 nM BSM; | 37 | 37 | 100% |
| 16-day | | | |
| embryos | | | |

In summary, the in vitro herbicidal selection system for the AIR herbicidal tolerance trait uses immature embryos harvested 12 or 14 days after pollination, depending on genotype, cultures on 5 nM metsulfuron-methyl or 300 nM bensulfuron-methyl, and 10 days in tissue culture without sub-culturing. With this system, described in the Sunflower AIR Trait in vitro Herbicidal Selection Protocol below (Example 11), 98% to 100% of trait-positive plants identified by visual screening using in vitro selection were confirmed trait-positive by TaqMan analysis across 6 AIR trait genotypes (Table 28).

TABLE 28
Percentage of AIR trait-positive plants selected from in vitro
culture using 300 nM bensulfuron-methyl (BSM) and 5 nM metsulfuron-
methyl (MSM), as confirmed by TaqMan analysis.

| | | % of AIR trait |
|---|---|---|
| | | positive plants |
| | | from in vitro selection |
| Herbicide | Treatment* | confirmed by TaqMan |
| BSM | 67/300 nM/12 day | 100 |
| | 3T/300 nM/12 day | 100 |
| | 3W/300 nM/12 & 14 day | 100 |
| | 3V/300 nM/14 day | 100 |
| | 3U/300 nM/14 day | 100 |
| MSM | 67/5 nM/12 day | 100 |
| | 3T/5 nM/12 day | 100 |
| | 3W/5 nM/12 & 14 day | 98 |
| | 3V/5 nM/14 day | 98 |
| | 3U/5 nM/14 day | 100 |

*The format of the listed treatments are as follows: AIR trait embryo name/BSM or MSM concentration/embryo age (e.g. 67/300 nM/12 day)

### Example 9. Sunflower Embryo Rescue Protocol

Immature embryo age was determined to standardize immature embryo parameters and reduce observed variation for embryos from different rows in the flower.

Step 1: Harvest immature sunflower seeds

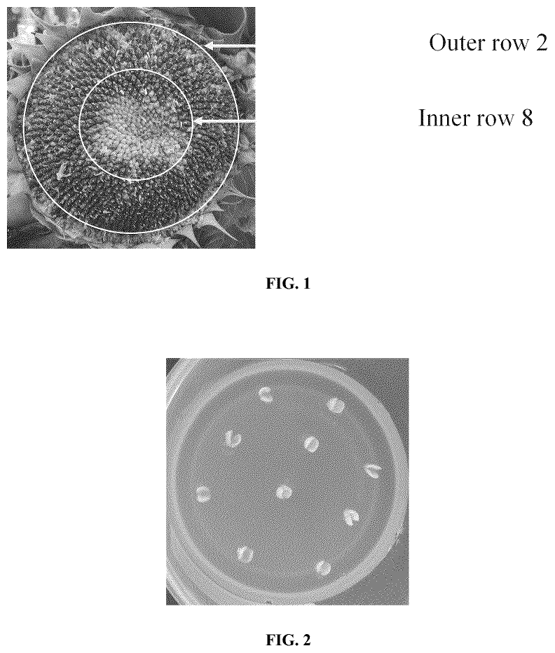

Harvest immature sunflower seed from self-pollinated or crossed florets (see method for harvesting sunflower immature embryos in example 10) 12 or 14 days after pollination (see FIG. 1), depending on genotype, from healthy plants in the greenhouse, removing the seed from the head. Note: immature embryo quality has a large effect on the growth and survival of the plantlets generated by embryo rescue (ER).

Step 2: Sterilize immature seeds in a laminar flow hood using good plant tissue culture sterile technique
1). Rinse immature seed with 75% ethanol, shaking by hand for 1 minute.
2). Soak seed in sterilizing solution (10% sodium hypochlorite solution, Sigma catalog #239305 500 ml; Chlorine 4-4.99%) with 1 drop of Tween 20 per 50 ml sterilizing solution for 15 minutes with shaking at 140-150 rpm.
3). Rinse seed with sterile water 6 times to remove residual sterilizing solution.

Step 3: Isolate immature embryos in a laminar flow hood using good plant tissue culture sterile technique
1). Coat new nitrile gloves (Micro-Touch, NitraTex) with 75% ethanol and allow to air dry in the laminar flow hood before putting them on.
2). Remove top or bottom of seed pericarp by hand. Then, squeeze out immature embryos and place them onto 90×15 mm petri dishes with 30 ml SF Germ 2 medium (see FIG. 2 and example 12) per plate to avoid drying out of embryos during extraction.
3). Allow immature embryos to remain in the plate for approximately 30 min to facilitate opening of the 2 cotyledons once extractions have been completed. This allows for easier placing on media in the next step. Note: it is important to ensure embryo coat removal to allow the embryo to germinate.

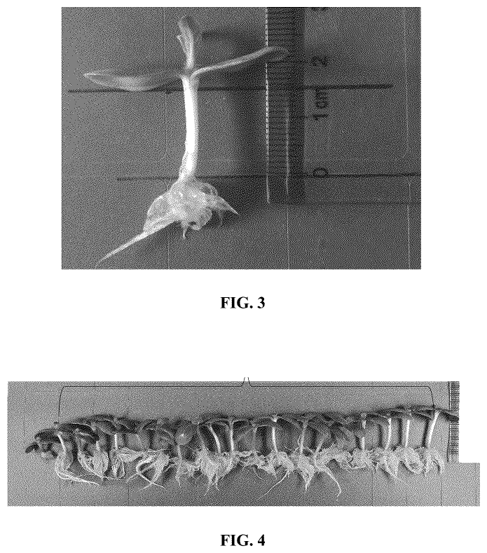

Step 4: Culture immature embryos in a laminar flow hood using good plant tissue culture sterile technique
1). Place 10 isolated immature embryos per 88 mm diameter×76 mm high clear plastic culture boxes with 100 ml of SF Germ 2 medium (example 12). Note: approximately ⅓ of immature embryo should be inserted into the medium, root first.
2). Place culture boxes with immature embryos in growth chamber at 25° C.+/−1° C. and a 16/8 light/dark photoperiod with 7K-8K lux illumination (Philips Lifemax cool light bulbs) for 7 days.
3). After 7 days, select all healthy ER plantlets for transplanting to the greenhouse. Healthy ER plants are defined as having a shoot height ≥1.5 cm (from root crown to cotyledon; see FIG. 3) with 2-4 main roots plus lateral roots and true leaves (see FIG. 4).

Step 5: Grow in vitro-generated ER plantlets in greenhouse
1). Transplant all healthy ER plants into 32 cell (7×7 cm per cell) propagation trays 7 days after tissue culture and place in a growth chamber for 2 weeks. Cover trays with a clear plastic dome immediately after transplanting. Maintain growth chamber temperature at 25° C. (day) and 15° C. (night) with a light intensity of 300 μmol/m²/s in 16-hour/8-hour (light/dark) photoperiod and a relative humidity of 40-60%. Use a standard seedling soil mix supplemented with 10 g Osmocote fertilizer per tray. Lighting in growth chamber uses Philips LED light bulbs with a 4.59:1 red:blue light ratio (4 bulbs with red, blue and far red, and 5 bulbs with red and white). Note: Ensure cleanliness of trays, domes and soil mix to prevent microbial contamination; transplant only healthy ER plants and cover with domes immediately after transplanting to ensure survival.
2). Remove domes 3-4 days after transplanting, depending on genotype (i.e. after seeing new leaf growth).
3). Water ER plants by hand as needed (typically, every other day) from the 2nd week on after removing domes.
4). Transplant ER plants from propagation trays to 5-gallon plastic soil pots in the greenhouse approximately 2 weeks after growth in chamber. Grow ER plants in the greenhouse under 26° C. (day) and 16° C. (night) temperatures, with a light intensity of approximately 15K lux and a relative humidity of 30-50% under a 14-hour/10-hour light/dark photoperiod. Use a standard soil mix supplemented as needed.
5). Fertilize ER plants immediately after transplanting into 5-gallon pots by spreading 45 g of Osmocote on the soil mix surface (plants are usually fertilized only once in greenhouse until harvest).
6). Watering regime:
   a. Hand water ER plants immediately after transplanting into 5-gallon pots. Note: Make sure to water thoroughly to ensure high soil moisture.
   b. Water ER plants every other day (600 ml water per pot) until R1 stage.
   c. Water ER plants every other day (1-2 L water per pot) at stages R1-R7.
   d. Water ER plants every other day (600 ml water per pot) at stages R7-R8.
   e. Stop water at the end of stage R8 to maturity.
   Note: Make sure to provide enough water for ER plants before the flowering and milk stages, monitoring and taking care of these ER plants every other day.

### Example 10. Harvesting Sunflower Immature Embryos

For CMS lines, harvest immature seeds 14 days after hand-pollination. Some genotypes allow for harvest at 12 days post pollination. For selfed or crossed lines, day 1 for self-pollination is when flowering of some disk florets in the first outer row is observed. Day 2 for self-pollination is the next day when flowering of most disk florets from outer rows 2-4 (˜3 rows). Day 3 and subsequent days are determined in the same way. Harvest immature seeds every 3 rows 14 days after each of day 1, day 2 and so on.

Note: To obtain immature embryos of relatively uniform age, critical for successful embryo rescue (ER; i.e. ≥85% of immature embryos producing healthy roots), we first identified the flowering pattern for 6 different genotypes to determine the self-pollination day. We usually see flowering of some disk florets of the 1st outermost row (=Day 1). Flowering of most disk florets in next 2-3 rows (outer rows 2-4) occurs simultaneously the day following Day 1 (=Day 2) and so on for Day 3 and subsequent days, until Day 7 or 8 depending on genotype. Usually disk florets flower in early morning and self-pollination occurs in late morning or early afternoon. This indicates that disk florets flower and self-pollinate in sequence, 2-3 rows every day after Day 1 and subsequent days. We do not harvest immature seeds in outer row 1 due to their nonuniformity. Therefore, immature seeds of 3 outer rows can be harvested 14 days after day 1, the next 2-3 rows 14 days after day 2, and so on.

### Example 11. Sunflower AIR Trait In Vitro Herbicidal Selection Protocol

Steps 1-3: refer to Sunflower Embryo Rescue Protocol (Example 9) and harvesting sunflower immature embryos above (Example 10).

Step 4: Culture immature embryos in a laminar flow hood using good plant tissue culture sterile technique
1) For in vitro herbicidal selection of the AIR trait, bensulfuron-methyl at 300 nM (medium BSM5) or metsulfuron-methyl at 5 nM (medium MSM5) are used. Medium SF germ5 is used as the herbicide control medium. All media recipes are in example 12 below. Stock solution for bensulfuron-methyl is made by dissolving 50 mg bensulfuron-methyl in 10 ml of DMSO (5 mg/ml) in a sterile tube. Stock solution for metsulfuron-methyl is made by dissolving 1 mg metsulfuron-methyl in 10 ml of DMSO (0.1 mg/ml) in a sterile tube. In both cases, 1 ml of stock solution is aliquoted into 2 ml sterile tubes and stored at −20° C.

Place 10 isolated immature embryos per 88 mm diameter×76 mm high clear plastic culture boxes with 100 ml of appropriate medium as described (example 12; FIG. 2). Note: approximately ⅓ of the immature embryo should be inserted into the medium, root first.
2) Place culture boxes with immature embryos in a growth chamber at 25° C.+/−1° C. with a 16/8 light/dark photoperiod with 7K-8K lux illumination (Philips Lifemax cool light bulbs) for 10 days.
3) After 10 days, select all healthy, herbicide tolerant plantlets for transplanting to the greenhouse. Healthy was defined as having a shoot height ≥2.5 cm from root to shoot tip and with 1-3 main roots (root length ≥1 cm) plus lateral roots.

Step 5: refer to Sunflower Embryo Rescue Protocol above.

### Example 12. Media Recipes

1. SF Germ 2 Media:

To approximately 500 to 600 ml Millipore pure water, add (while stirring) 2.15 g MS basal salts and 20 g sucrose. Bring to 1 L with nano-pure water and adjust the pH to 5.7 with KOH. Add 8 g agar and autoclave for 20 minutes. Allow it to cool to 48° C.-50° C. in a sterile laminar flow hood before pouring into culture box using sterile technique.

2. SF Germ 5 Media:

This is SF Germ 2 media with the addition of 0.5 g/L MES (C6H13NO4S) before autoclaving.

3. BSM5 Media:

This is SF Germ 5 media with the addition of 25 uL sterile 5 mg/ml bensulfuron-methyl solution after autoclaving.

4. MSM5 Media:

This is SF Germ5 media with the addition of 19 uL sterile 0.1 mg/ml metasulfuron-methyl solution after autoclaving.

What is claimed is:

1. A method for a plant embryo rescue, the steps comprising:
a. obtaining immature seeds at 20 or fewer days after pollination (“DAP”);
b. isolating immature embryos from the immature seeds of step a.;
c. culturing the immature embryos of step b.; and
d. growing plantlets from the cultured immature embryos of step c. in suitable growth media and conditions;
wherein the suitable growth media comprises a sucrose concentration, a photoperiod, and a temperature.

2. The method of claim 1, wherein the immature seeds of step a. are sterilized in ethanol or a sterilizing solution comprising hypochlorite and/or bleach.

3. The method of claim 1, wherein the immature seeds are a dicot species.

4. The method of claim 3, wherein the dicot species is sunflower.

5. The method of claim 1, wherein the immature embryo age for enhanced plantlet development is 10 to 18 days after pollination.

6. (canceled)

7. (canceled)

8. The method of claim 1, wherein the sucrose concentration for embryo growth is 15 g/l to 30 g/l.

9. (canceled)

10. The method of claim 1, wherein the photoperiod for embryo growth is (i) 16-hour day/8-hour night, (ii) 8-hour day/16-hour night, (iii) 5 day night plus 16-hour day/8-hour night for 2 days, or (iv) 3 days night plus 16-hour day/8-hour night for 4 days.

11. (canceled)

12. (canceled)

13. The method of claim 1, wherein the temperature for embryo growth is 20° C. to 30° C.

14. (canceled)

15. A plant, plant part, or progeny thereof, wherein the plant, plant part, or progeny thereof is produced by the method of claim 1.

16. A method for in vitro herbicide resistance trait selection using the embryo rescue methodology of claim 1, wherein the immature embryos are grown on a culture medium comprising an herbicide, wherein the herbicide is selected from the group consisting of imidazolinones, pyrimidinylthiobenzoates, sulfonylaminocarbonyltriazolinone, sulfonylureas, triazolopyrimidines, amino acid derivatives, isoxazoles, pyrazolones, or triketones.

17. (canceled)

18. The method of claim 16, wherein the trait is selected from the group consisting of the Ahasl1, ESPS, PAT, or an HPPD-inhibitor resistance gene.

19. The method of claim 18, wherein the trait is the AIR herbicidal tolerance trait.

20. The method of claim 16, wherein the culture medium comprises bensulfuron-methyl (BSM) or metsulfuron-methyl (MSM).

21. The method of claim 16, wherein the immature embryos are harvested 10 to 18 days post pollination.

22. (canceled)

23. (canceled)

24. The method of claim 16, wherein the immature embryos are in tissue culture for 5 to 10 days without sub-culturing.

25. (canceled)

26. (canceled)

27. The method of claim 16, wherein the method further comprises growing selected plantlets into plants and backcrossing with another plant to obtain another generation.

28. The method of claim 16, wherein the immature embryos are harvested from a dicot species.

29. (canceled)

30. The method of claim 20, wherein the culture medium BSM concentration is 100 nM to 500 nM.

31. (canceled)

32. The method of claim 20, wherein the culture medium MSM concentration is 1 nM to 10 nM.

33. (canceled)

34. A plant, plant part, or progeny thereof, derived from a plant, plant part, or progeny thereof produced by the method of claim 16.
